SEQUENCE LISTING (22 sequences)

SEQ ID NO: 1 - DNA, 623 bp, Artificial Sequence
  Synthetic U6-STOP-shA1 construct
tccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataacttcgtatagcatacattatacgaagttattacgtttttgcgatttttgaattcgttcctcagaggaactgacaagcaccctaacatcctattggaggctcactcacgttttttctattttgtttcttgacagcagagctcgttgctcactgtatagctcaggttggcctgacactgatgaggttctccagtgactgcctctacctacctactgggatgacagaggtgtaccaccaagccacgcccgggggatccataacttcgtatagcatacattatacgaaggaaatgctctttctcctcaaagctttgaggagaaagagcatttcccttttt

SEQ ID NO: 2 - DNA, 282 bp, Artificial Sequence
  Functional units of the U6-STOP-shA1 construct
tccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagac

SEQ ID NO: 3 - DNA, 5 bp, Artificial Sequence
  U6 promoter of TATA box
tataa

SEQ ID NO: 4 - DNA, 34 bp, Unknown
  Wild type of loxP sequence
ataacttcgtatagcatacattatacgaagttat

SEQ ID NO: 5 - DNA, 225 bp, Artificial Sequence
  Stop casete sequence includes U6 po1 III termination
tacgtttttgcgatttttgaattcgttcctcagaggaactgacaagcaccctaacatcctattggaggctcactcacgttttttctattttgtttcttgacagcagagctcgttgctcactgtatagctcaggttggcctgacactgatgaggttctccagtgactgcctctacctacctactgggatgacagaggtgtaccaccaagccacgcccgggggatcc

SEQ ID NO: 6 - DNA, 212 bp, Artificial Sequence
  genomic U6 PolIII termination sequence
tttttgaattcgttcctcagaggaactgacaagcaccctaacatcctattggaggctcactcacgttttttctattttgtttcttgacagcagagctcgttgctcactgtatagctcaggttggcctgacactgatgaggttctccagtgactgcctctacctacctactgggatgacagaggtgtaccaccaagccacgcccgggggatcc

SEQ ID NO: 7 - DNA, 34 bp, Artificial Sequence
  the mutant second loxP site downstream of the STOP cassette
ataacttcgtatagcatacattatacgaaggaaa

SEQ ID NO: 8 - DNA, 22 bp, Artificial Sequence
  Primer
ggacctccatctgctcttattt

SEQ ID NO: 9 - DNA, 22 bp, Artificial Sequence
  Primer
ggtctattactgtgcaagttgg

SEQ ID NO: 10 - DNA, 27 bp, Artificial Sequence
  Primer
tgtgaattcgttcctcagaggaactga

SEQ ID NO: 11 - DNA, 36 bp, Artificial Sequence
  Primer
tgtggatcccccgggcgtggcttggtggtacacctc

SEQ ID NO: 12 - DNA, 29 bp, Artificial Sequence
  Primer
gactctagatccgacgccgccatctctag

SEQ ID NO: 13 - DNA, 85 bp, Artificial Sequence
  Primer
tgcgaattcaaaaatcgcaaaaacgtaataacttcgtataagtatgctatacgaagttatagtctcaaaacacacaattacttac

SEQ ID NO: 14 - DNA, 35 bp, Artificial Sequence
  Primer
tgctcgagatgtctgagtacgagttcatgcatatc

SEQ ID NO: 15 - DNA, 41 bp, Artificial Sequence
  Primer
ctggatccttatttcagcaggaacagcatctcccatatctg

SEQ ID NO: 16 - DNA, 32 bp, Artificial Sequence
  Primer
ctggatccttacttgaggagaaagagcatttc

SEQ ID NO: 17 - DNA, 21 bp, Artificial Sequence
  Primer
ttcctaataacccagcctttg

SEQ ID NO: 18 - DNA, 21 bp, Artificial Sequence
  Primer
gtgatggcaggagatttgtaa

SEQ ID NO: 19 - DNA, 24 bp, Artificial Sequence
  Primer
cattaactggggaaggattgtgac

SEQ ID NO: 20 - DNA, 24 bp, Artificial Sequence
  Primer
gcagaaaagtcagccagccagatt

SEQ ID NO: 21 - DNA, 20 bp, Artificial Sequence
  Primer
caagagggagagcaagccta

SEQ ID NO: 22 - DNA, 20 bp, Artificial Sequence
  Primer
cgtctcaggccttcagtgag

(19) United States
(12) Patent Application Publication
(10) Pub. No.: US 2008/0153764 A1
(43) Pub. Date: Jun. 26, 2008

(54) System and Methods For Short Rna Expression

(75) Inventors: Philipp Oberdoerffer, Boston, MA (US); Chrysi Kanellopolou, Brookline, MA (US)

Correspondence Address: FISH & RICHARDSON PC, P.O. BOX 1022, MINNEAPOLIS, MN, 55440-1022, US

(73) Assignee: THE CBR INSTITUTE FOR BIOMEDICAL RESEARCH, INC., Boston, MA (US)

(21) Appl. No.: 10/585,886

(22) PCT Filed: Jan. 21, 2005
(86) PCT No.: PCT/US05/03104

§ 371 Date: Jun. 25, 2007

Related U.S. Application Data
(60) Provisional application No. 60/538,871, filed on Jan. 22, 2004.

(51) Int. Cl. A61K 31/70; C07H 21/02; A01K 67/027; C12N 5/10; A61P 43/00; C12N 15/87; C12Q 1/68

(52) U.S. Cl. 514/44; 536/24.1; 800/8; 800/17; 800/18; 800/16; 800/15; 800/14; 435/366; 435/325; 536/25.3; 800/21; 435/6; 435/455; 800/3

(57) ABSTRACT

The invention provides inducible expression systems for making short RNA transcripts that can be used in cells and transgenic animals for a variety of applications, including but not limited to, producing and studying the effects of RNAi and microRNA mediated gene silencing.

### STATEMENT AS TO FEDERALLY SPONSORED RESEARCH

This invention was made with government support under grant number P01 AI56900 awarded by NIH/NIAID.

### TECHNICAL FIELD

This invention relates to technologies for regulating gene expression, and more particularly to inducible systems for expressing short RNA molecules.

### BACKGROUND

RNA interference (RNAi) is a powerful and widely used method to inhibit gene product expression in model organisms. RNAi is a highly coordinated post-transcriptional mechanism that was first described in nematodes. In RNAi, long double stranded RNAs and complex hairpin RNAs are processed into small interfering RNAs (siRNAs). These siRNAs are generally 21-23 bp RNA duplexes with characteristic dinucleotide overhangs. Duplex siRNAs are processed by helicases into single stranded siRNAs, which are able to participate in RNA induced silencing complexes (RISC). The RISC complex functions as a highly specific endonuclease that usually cleaves target RNAs with perfect complementarity to the siRNA in the RISC complex.

The power of RNAi as a tool lies in two features of the reaction just described. First, siRNAs trigger a self-amplifying feedback loop that requires only a small number of initial siRNAs to potentially degrade a large number of target RNAs. Cleavage of target RNAs by a RISC complex generates additional single stranded siRNAs, which in turn are able to participate in additional RISC complexes. Second, RNAi exhibits exquisite specificity. A single base pair mutation in either the siRNA, or in the target RNA, typically prevents RNAi silencing of the target RNA expression.

The power of siRNAs has fostered interest in the development of systems that can be used for RNAi-mediated silencing of pre-selected target genes in mammalian cells. Some systems employ chemical or enzymatically synthesized siRNAs to transiently induce RNAi in cells. Other systems use plasmid and viral vectors to express hairpin RNAs (siRNA-like transcripts) to stably induce the knockdown of expression of pre-selected genes. See, e.g., Brummelkamp, et al., Science 296:550-553 (2002) and Novina, et al, Nat Med 8, 681-686 (2002); Rubinson, et al, Nat. Genet. 33:401-406 (2003). A third class of systems employ technologies that allow for conditional expression of siRNA-like transcripts. Czauderna, et al., Nucleic Acids Res 31:e12 (2003) and Kasim, et al, Nucl. Acid. Res. Supp. No 3: 255-256 (2003).

### SUMMARY OF THE INVENTION

The invention is based on novel expression systems that inducibly produce short RNA transcripts. The short RNA expression systems described herein have the ability to inducibly and very precisely, e.g., without extraneous sequence, produce short RNA transcripts, whose sequences can be pre-selected. These short RNA expression systems are very well suited for expressing RNA transcripts that are designed to induce gene silencing via any of the gene silencing mechanisms known to operate through very short, and often highly specific, RNA molecules. The invention also provides transgenic animals and cells carrying the short RNA expression systems disclosed herein. Because the systems of the present invention are inducible, they can be used to study the role of essential genes in cells and animals in ways that are not possible in constitutive expression systems. Additionally, the inducible expression system of the present invention can be used to study the effects of induced gene silencing in specific tissues.

In general, the invention features a nucleic acid molecule that includes the following sequence components: a promoter sequence capable of transcribing short RNA transcripts, a short RNA encoding sequence that encodes a short RNA transcript, and a STOP cassette.

Short RNA transcripts are transcripts with, e.g., fewer than 400 bases, or fewer than 201 bases, or fewer than 150 bases, or fewer than 100 bases, or fewer than 50 bases. Short RNA transcripts include RNA molecules capable of eliciting RNAi-mediated or micro-RNA-mediated gene silencing.

A STOP cassette includes the following sequence components: a termination sequence capable of preventing or terminating transcription by the RNA polymerase that binds the promoter sequence, a first loxP sequence, and a second loxP sequence. The loxP sequences flank the termination sequence. The termination sequence is positioned along the nucleic acid between the promoter sequence and the transcription initiation site of the short RNA encoding sequence in the nucleic acid molecule. In some, but not all, embodiments the short RNA encoding sequence overlaps with one of the loxP sequences.

In a first aspect, the invention features a nucleic acid molecule that includes: an RNA polymerase III promoter sequence; a short RNA encoding sequence that includes a transcription initiation site; and a STOP cassette. The STOP cassette includes an RNA polymerase III-specific termination sequence, a first loxP sequence and a second loxP sequence. The loxP sequences flank the termination sequence, and the termination sequence is disposed between the promoter sequence and the transcription initiation site of the short RNA encoding sequence in the nucleic acid molecule. In some, but not all, embodiments the short RNA encoding sequence overlaps with one of the loxP sequences.

In some embodiments of the first aspect, the first loxP sequence is a wild-type loxP sequence. In some embodiments of the first aspect, the second loxP sequence is the loxP that is downstream from the termination sequence, and the second loxP is a mutant loxP sequence. For example, the second loxP sequence can contain sequence that overlaps with some or all of the short RNA encoding sequence. In other words, the n-terminal nucleotides in the terminus of the loxP that is proximal to the short RNA consists of the 5′ terminal sequence of the short RNA encoding sequence, wherein n=1 to 10. In other examples of this embodiment, the five terminal nucleotides in the loxP sequence overlap with, i.e. consist of, the five 5′ terminal nucleotides of the short RNA encoding sequence. The five 5′ terminal nucleotides of the short RNA encoding sequence is the sequence that includes the (+1) through (+5) positions of the transcript encoding sequence.

In some embodiments of the first aspect, the nucleic acid includes a thymidine nucleotide in the sequence position that immediately precedes the upstream terminal sequence of the loxP sequence that is located upstream of the termination sequence. An example of this embodiment also includes the wild-type first loxP sequence described above. Some examples of this embodiment also include the mutant second loxP sequences described above, i.e. in which the n-terminal nucleotides in the terminus of the loxP that is proximal to the short RNA consists of the 5′ terminal sequence of the short RNA encoding sequence, wherein n=1 to 10.

In some embodiments of the first aspect, the promoter sequence includes some portion of the RNA polymerase III promoter sequence from the genomic sequence of the small nuclear RNA U6 promoter. Examples of this embodiment include nucleic acids with a STOP cassette that includes, from 1-190 bases of the genomic sequence that is immediately downstream of the small nuclear RNA U6 genomic transcription termination signal. In another example of this embodiment, the STOP cassette of the nucleic acids include a modified genomic U6 transcription termination sequence that includes: some number, from 1 to 20, inclusive, of additional thymidine nucleotides disposed immediately adjacent to the wild-type U6 thymidine termination signal (or T-stretch); and also includes some number, from 1 to 190, inclusive, of nucleotides encoding the wild-type U6 genomic sequence that is immediately downstream of the thymidine termination sequence. In some examples of this embodiment, the termination sequence includes more than one T-stretch and also includes some number, from 1 to 190, inclusive, of nucleotides encoding the wild-type U6 genomic sequence that is immediately downstream of the thymidine termination sequence. Some examples of this embodiment also include a wild-type loxP sequence. Some examples of this embodiment also include the mutant loxP sequences described above, i.e. in which the n-terminal nucleotides in the terminus of the loxP that is proximal to the short RNA consists of the 5′ terminal sequence of the short RNA encoding sequence, wherein n=1 to 10.

In other embodiments of the first aspect, the short RNA encoding sequence encodes a transcript with fewer than 400, e.g., fewer than 200, fewer than 100, fewer than 70, fewer than 60, fewer than 50, fewer than 40, or fewer than 30 nucleotides. Examples of this embodiment also include one or more of the following: any of the promoter sequences, any of the termination sequences, the wild-type loxP sequence, or any of the mutant loxP sequences that are described herein.

In a second aspect, the invention features a transgenic animal that has incorporated into its genome any of the nucleic acids described herein, for example the nucleic acids described in the first aspect of the invention.

In one embodiment, the transgenic animal also includes a nucleic acid molecule encoding a Cre recombinase. In one example of this embodiment, expression of the Cre recombinase is developmentally regulated, e.g., the Cre recombinase is maximally expressed only at one or more specific stages of embryonic or animal development. In another example of this embodiment, expression of the Cre recombinase is tissue-specific, e.g., the Cre recombinase is maximally expressed only in one or more specific cell types.

In some embodiments, the transgenic animal described herein is one of the following: a mouse, a rat, a goat, a pig, a monkey, a cow; a rabbit; a sheep, a hamster, a chicken, or a frog. In one example of this embodiment, expression of the Cre recombinase is developmentally regulated, e.g., the Cre recombinase is maximally expressed only at one or more specific stages of embryonic or animal development. In another example of this embodiment, expression of the Cre recombinase is tissue-specific, e.g., the Cre recombinase is maximally expressed only in one or more specific cell types.

In a third aspect, the invention features a eukaryotic cell that includes any of the nucleic acids described herein, for example, the nucleic acids described in the first aspect of the invention. In one embodiment, the cell is an animal cell, e.g., the cell is a mammalian cell. In another embodiment the cell is an embryonic stem cell.

In some embodiments, any of the cells described herein also includes a nucleic acid molecule encoding a Cre recombinase gene. In other embodiments, any of the cells described herein also include a Cre recombinase protein.

In a fourth aspect, the invention features a method of making an inducible short RNA expression system. The method includes linking two or more nucleic acids to produce any one of the nucleic acids described herein, e.g., the nucleic acids described in the first aspect of the invention.

In a fifth aspect, the invention features a method of making a transgenic animal. In one embodiment, the method includes introducing into the genome of an embryonic stem (ES) cell any of the nucleic acid molecules described herein, e.g., the nucleic acids described in the first aspect of the invention, to generate a transgenic ES cell. The method also includes introducing the transgenic ES cell into an embryo, implanting the embryo into an animal capable of carrying the embryo to term, and allowing the embryo to come to term, thereby generating a transgenic animal. In one example of this embodiment, the method generates a chimeric transgenic animal, and the method further includes crossing the chimeric transgenic animal to another animal of the same species to generate a founder transgenic animal.

In another embodiment, the method includes introducing into the genome of an oocyte any of the nucleic acid molecules described herein, e.g., the nucleic acids described in the first aspect of the invention. The method also includes fertilizing the oocyte to produce an embryo, implanting the embryo in an animal capable of carrying the embryo to term, and allowing the embryo to come to term, thereby generating a transgenic animal.

In a sixth aspect, the invention features a method of making an animal cell containing an inducible short RNA expression. The method includes transfecting a cell with any of the nucleic acid molecules described herein, e.g., the nucleic acids described in the first aspect of the invention. In an example of the method, the transfected cell is a cell from any one of the following animals: a human, a mouse, a rat, a goat, a pig, a monkey, a cow; a rabbit; a sheep, a chicken, a frog, or a fish.

In a seventh aspect the invention features a method of studying gene function in a cell. The method includes: providing any of the cells described herein, e.g., the cells of the third aspect, inducing transcription of the short RNA encoding sequence; and monitoring changes in the cell.

In an eighth aspect, the invention features a method of studying gene function in an organism. The method includes: providing any of the transgenic animals described herein, e.g., the transgenic animals described in the second aspect of the invention, inducing transcription of the short RNA encoding sequence; and monitoring changes in the organism.

### TERMS

“Short RNAs” and “short RNA transcripts” are ribonucleic acids, typically less than 400 bases in length. Some short RNAs are capable of eliciting RNAi-mediated or Micro-RNA-mediated gene silencing.

“Short RNA encoding sequence” is a nucleic acid sequence coding for a short RNA transcript. Typically a short RNA encoding sequence will be a DNA sequence coding for a short RNA transcript. A short RNA encoding sequence can also be an RNA sequence, e.g., in an RNA virus vector, that encodes, e.g., by reverse transcription, a short RNA transcript.

“Transcription unit” is a nucleic acid that includes a promoter sequence, a transcript sequence, and a transcript termination sequence.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE FIGURES

FIG. 1(a) and (b) are schematic diagrams of the U6lox-shA1 construct (a) before a Cre-mediated excision of the termination sequence, and (b) after a Cre-mediated deletion of the termination sequence.

FIG. 2(a) is a diagram of the targeting strategy for inserting the U6lox-shA1 construct into the HPRT locus of HM1 stem cells. FIG. 2(b) is a Southern Blot confirming insertion of the U6lox-shA1 construct.

FIG. 3 is a schematic diagram of the A1-IRES-EGFP reporter construct.

FIG. 4(a)-(d) are the results of experiments verifying the Cre-mediated induction of shA1 expression and subsequent specific downregulation of the A1-IRES-EGFP reporter construct.

Like reference symbols in the various drawings indicate like elements.

### DETAILED DESCRIPTION

The following is a description of specific embodiments of the invention. The inducible short RNA expression systems and methods are described in conjunction with specific nucleic acid sequences. Nevertheless, it should be recognized that the inducible expression system and methods described in the present specification and the claims may also be used in conjunction with other nucleic acid sequences. Although the inducible short RNA expression systems are described as useful in methods that regulate gene expression through RNAi and micro-RNA induced mechanisms, it should be recognized that the systems are also useful in other methods, e.g. in applications that require the expression of short RNAs for purposes other than RNA-mediated gene product regulation.

### Brief View of the Novel Expression Systems

The components of the expression systems include an RNA Polymerase III (Pol III)-specific promoter sequence, a loxP-flanked STOP cassette sequence, and a short RNA encoding sequence. These three nucleotide sequences are arranged on a nucleic acid such that the promoter is upstream of the STOP cassette, and STOP cassette is upstream of the short RNA encoding sequence. The terms upstream and downstream as used herein refer to the direction of productive transcription on a nucleic acid molecule starting from the Pol III promoter's transcription start site. Productive transcription starts from an upstream position on a nucleic acid molecule and proceeds downstream along the molecule, until transcription is terminated. Thus, in the present systems, the short RNA encoding sequence is downstream of the STOP cassette, and the STOP cassette is downstream of the Pol III-specific promoter. The relative locations of these three components in the present system prevents transcription of the short RNA encoding sequence by RNA polymerase III because the STOP cassette's termination sequence is located between the Pol III promoter and the short RNA encoding sequence.

When Pol III polymerase assembles on the Pol III promoter sequence of the systems, it proceeds downstream from the promoter sequence towards the short RNA transcript. Before it reaches the short RNA encoding sequence, though, the polymerase encounters the termination sequence in the STOP cassette. The termination sequence causes the polymerase to abort the transcription reaction before any short RNA encoding sequence is transcribed.

Transcription of short RNA transcripts in the systems can be induced by causing the nucleic acid to be contacted by a Cre recombinase. Cre recombinase can catalyze the excision of the STOP cassette from the nucleic acid, thereby producing a nucleic acid that no longer contains a transcription termination signal between the promoter sequence and the short RNA encoding sequence. Cre-mediated excision of the STOP cassette in the present systems modifies the nucleic acids of the systems disclosed herein to allow Pol III promoter driven transcription of the short RNA encoding sequence.

### Detailed View of the Nucleic Acids of the Novel Short RNA Expression Systems

### 1. Promoter Sequences

Promoters that can be used in the short RNA expression system of the present invention are nucleic acids that include a promoter sequence capable of driving expression of short RNAs, e.g., RNAs which can induce RNAi or micro-RNA mediated gene silencing. Preferred promoters are those whose transcription start and stop sites are very predictable and precise. Examples of such promoters are the RNA polymerase III (Pol III)-specific promoters, which include the Pol III type 3 core promoters, which are described in detail in Schramm and Hernandez, Genes & Dev. 16:2593-2620 (2002). Pol III promoter sequences are DNA sequences that recruit Pol III, i.e. on which Pol III can assemble inside of a cell, for the first step of a Pol III transcription reaction.

Promoters that can be used in the present invention can include the U6 snRNA gene (U6) promoter sequence. The U6 gene is transcribed by Pol III and encodes the U6 snRNA component of the splicesosome. The U6 promoter sequence can be the U6 promoter sequence from a mammal, including a human or a mouse, or it can be the U6 promoter sequence from a non-mammalian animal. Other Pol III promoters that can be used in the present invention include promoter sequences that drive transcription of the III RNAse P gene (H1). The H1 promoter sequence can be the sequence of the H1 promoter from a human, a mouse, a mammal, or an animal.

The U6 and H1 Pol III transcription units share several unusual features. First, none of the promoter elements, except the (+1) transcription start site, is located in the transcribed region of either the U6 or H1 gene. This feature means that almost any pre-selected sequence can be placed downstream the U6 or H1 promoter start site, and Pol III will drive expression of that sequence. Second, Pol III promoters, e.g., the U6 and H1 promoters, start transcription from precisely defined distances, i.e., between 32 and 25 bp, downstream of the TATA box. This feature provides the necessary control for the expression of short pre-selected transcripts. Third, Pol III recognizes a run of 4-5 thymidine residues as a termination signal. This feature not only allows for easy control of transcript termination, but also results in overhanging uridines, which resembles the overhanging uridines or thymidines at the end of synthetic siRNAs. Finally it is worth noting that Pol III normally transcribes only very short genes, generally less than 400 bp.

### 2. The STOP Cassette

The STOP cassettes of the present invention are nucleic acids. The nucleotide sequence of these nucleic acids includes: a transcription termination sequence and two loxP sequences. The two loxP sequences flank the termination sequence, i.e., one loxP is positioned at the 5′ terminus of the termination sequence, (i.e. upstream of the termination sequence) and the other loxP is positioned at the 3′ terminus of the termination sequence (i.e. downstream of the termination sequence).

The choice of termination sequence used in a STOP cassette will depend on the polymerase activity the STOP cassette is designed to terminate. Thus, if the promoter sequence used in a system of the present invention is a Pol III promoter sequence, then the termination sequence used in the system is a sequence capable of preventing or terminating Pol III transcription. If the promoter sequence is one that recruits another kind of polymerase, then the transcription termination sequence of the STOP cassette is a sequence capable of preventing or terminating transcription of that other kind of polymerase that is recruited by the promoter.

The Pol III polymerase is unique in its ability to recognize a simple run of four to five consecutive thymidines as a termination signal (T-stretch). Schramm and Hernandez, Genes & Dev. 16:2593-2620 (2002). Transcription termination can be enhanced by including multiple T-stretches at the end of a Pol III transcribed gene. Transcription termination can also be enhanced by increasing the number of consecutive thymidines in a T-stretch. Furthermore, reports have also suggested that untranscribed sequence downstream of the termination signal can affect the termination efficiency of Pol III termination signal. Das, et al, EMBO J. 7:503-512 (1988).

When a Pol III promoter is used in a system of the present invention, appropriate termination sequences for use in the system can be sequences that include a run of four to five consecutive thymidines. The termination sequence can optionally include more than 5 consecutive thymidines. The termination sequence can optionally include untranscribed downstream sequences from known genomic Pol III termination signals.

For example, when a Pol III promoter is used in a system of the present invention, the termination sequence can include sequences that are downstream of the genomic U6 termination signal. The termination sequence can include any number, from 50 to 190, of bases of the wild-type genomic U6 sequence that is downstream of the U6 gene's T-stretch.

Other examples of termination sequences that can be used in conjunction with a Pol III promoter sequence in systems of the present invention can include sequences that are downstream of the H1 termination signal. The termination signal can include any number, from 20 to 190, of bases of the wild-type H1 sequence that is downstream of the H1 gene's T-stretch.

The loxP sequences in the STOP cassette can include wild-type loxP sequences or one or two mutant lox P sequences. Wild-type LoxP sequences are 34 base pair (bp) sequences that are recognized by the Cre recombinase in reactions described more fully below. A wild-type loxP sequence is consists of two 13 bp inverted repeats separated by an 8 bp spacer region. The loxP sequence has been published and is also provided in the Example below. See, e.g., Sauer, B., Nucl. Acids Res. 24:4608-4613 (1996). It is worth noting that to be functional, a wild-type loxP sequence must be on a double stranded DNA molecule. The systems of the present invention are not limited to double stranded DNA molecules. For example, the present invention contemplates the use of retroviruses that carry sequences coding for a promoter, a loxP-flanked terminator sequence, and a short RNA encoding sequence. Such retroviruses might be used to insert DNA molecules in the genome of a host, thereby generating a functional inducible expression system. The terms “wild-type loxP” sequence or “mutant loxP sequence” therefore should also be understood to include single stranded DNA sequences and RNA sequences coding for functional DNA loxP sequences.

In some embodiments the expression system of the present invention will include one mutant loxP sequence. The mutant loxP sequence can be the loxP sequence that is upstream or the loxP sequence that is downstream of the termination sequence in the STOP cassette. Some mutant loxP sequences will contain one or more mutated bases in the terminal 10 bases of one terminus of a loxP sequence. The terminus of a loxP sequence refers to one of the two 5′ and 3′ ends of the loxP sequence. Thus every loxP in a STOP cassette contains two termini, an upstream and a downstream terminus relative to the direction of productive transcription generated by the promoter sequence in the system. The terminal 10 bases of a loxP terminus are the ten consecutive bases that constitute one of the two termini of a loxP sequence.

In some embodiments the mutated loxP sequence will include one or more mutant bases in the downstream terminus of the loxP sequence that is downstream of the termination sequence. Examples of such mutants are loxP mutants are loxP sequences that contain one or more mutation in the 10 bases of the downstream terminus. In some examples the mutant downstream loxP terminal sequence will overlap with the first 1-10, e.g., 5, bases of the short RNA encoding sequence. In other words the downstream terminal sequence, of the loxP sequence located downstream of the termination sequence, can include, or overlap with, the upstream terminal sequence of the short RNA encoding sequence. The usefulness of such mutant loxPs is explained below.

### 3. Short RNA Encoding Sequences

The short RNA encoding sequences of the present invention are nucleic acid sequences coding for short RNA transcripts. Short RNA transcripts are transcripts consisting of 120 nucleotides or less. Short RNA encoding sequences include those that code for siRNA-like hairpins, which can be between 10 and 40 nucleotides in length. In some systems short RNA encoding sequences encode transcripts that are between 15 and 30 nucleotides in length. In some systems short RNA encoding sequences encode transcripts that are between 18 and 24 nucleotides in length. Many short RNA encoding sequences include sequences coding for transcripts that can activate a cell's RNAi gene silencing mechanisms.

Short RNA transcripts also include micro-RNA-like precursors and micro RNA-like transcripts. Micro-RNA precursors can be approximately 70 nucleotides in length. Lee et al, EMBO J. 21:4663-4670 (2002). Processed Micro RNAs can be much smaller, e.g., from 10-40 nucleotides long, or 15-30 nucleotides long, or most frequently between 18-24 nucleotides long. Micro-RNAs mediate gene-silencing through a different mechanism than RNAi. Unlike siRNAs MicroRNAs are not usually perfectly complementary to their targets. short RNA encoding sequences in the present system include sequences coding for transcripts that activate a cells micro-RNA mediated gene-silencing mechanisms.

In keeping with standard molecular biological usage, the first nucleotide of the short RNA transcript is encoded by the transcription initiation (+1) site of the short RNA encoding sequence. The transcription initiation site is therefore upstream of every other nucleotide in the short RNA encoding sequence. The second nucleotide in the short RNA encoding sequence that is transcribed can be referred to as the (+2) position, and the third nucleotide in a developing transcript is coded for by the (+3) position in the short RNA encoding sequence, etc.

In some embodiments the upstream portion of the short RNA encoding sequence overlaps with the closest, i.e., proximal loxP sequence in the nucleic acid. (The proximal loxP to the short RNA encoding sequence is the downstream loxP relative to the other loxP in the system). In these embodiments the downstream terminal sequence of the short RNA encoding sequence-proximal loxP sequence is the upstream sequence of the short RNA encoding sequence. Stated differently, the downstream terminal sequence of the downstream loxP contains the transcription initiation site of the short RNA encoding sequence, and optionally includes one or more bases of additional short RNA encoding sequence.

In some embodiments of the system, the 10 terminal bases of the downstream terminal of the downstream loxP sequence are also the +1 through +10 positions of the short RNA encoding sequence. In other embodiments 5 terminal bases of the downstream terminal of the downstream loxP sequence are also the +1 through +5 positions of the short RNA encoding sequence.

Termination of transcription of the short RNA encoding sequences is achieved by placing a termination signal immediately downstream of the short RNA encoding sequence. In the present system, the most downstream portion the short RNA encoding sequence will contain the first one, two, or three thymidines of the stretch of consecutive thymidines that represents a Pol III termination signal.

### Functional Equivalents

Skilled artisans will recognize that functional equivalents can be used in place of certain sequences described herein, in conjunction with the inducible expression systems disclosed herein. For example, in one embodiment, a functional equivalent can be used instead of the mouse genomic U6 promoter sequence provided in Table 2 of Example 1. Functional equivalents of the mouse U6 promoter sequence include sequences that differ by one or more bases from the sequence provided in Table 2 and that retain an ability to recruit RNA polymerase III in the first step of a reaction that leads to productive RNA transcription. Similarly, the functional equivalent of any other Pol III promoter sequence, e.g. the human genomic U6 promoter sequence, the human or mouse genomic H1 promoter sequences, include sequences that differ by one or more bases from the Pol III promoter sequences and also retain an ability to recruit Pol III in the first step of a reaction that leads to productive transcription.

Functional equivalents can also be used instead of genomic sequences downstream of a Pot III termination signal.

Functional equivalent sequences include those sequences that also have a high percentage of identity to the sequences already known to skilled artisans and/or those sequences disclosed herein that can be used in conjunction with the expression systems of the present invention. Functional equivalents include sequences with 99%, 98%, 97%, or any percentage higher than 90%, or any percentage higher than 80%, or any percentage higher than 70%, identity to a known or disclosed sequence.

To determine the percent identity of two nucleic acid sequences, the sequences are aligned for optimal comparison purposes (e.g., gaps can be introduced in one or both of a first and a second nucleic acid sequence for optimal alignment and non-homologous sequences can be disregarded for comparison purposes). In a preferred embodiment, the length of a reference sequence aligned for comparison purposes is at least about 30%, preferably at least about 40%, more preferably at least about 50%, even more preferably at least about 60%, and even more preferably at least about 70%, 80%, 90%, or 100% of the length of the reference sequence. The nucleotides at corresponding nucleotide positions are then compared. When a position in the first sequence is occupied by the nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 26493 nucleic acid molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

### Methods of Making the Nucleic Acid of the Present Invention

Techniques and methods for engineering recombinant nucleic acids are well known in the art. Examples of such techniques and methods include, enzymatic nucleotide restrictions, site directed mutagenesis, and in vitro transcription.

### Methods of Using the Nucleic Acids of the Present Invention

The nucleic acids of the present invention can be placed inside living cells and organisms. For example, the nucleic acids of the present invention can be placed in nucleic acid vectors which are subsequently introduced into hosts by a variety of methods which are known in the art, e.g., transformation, transfection, electroporation, and liposome delivery. Examples of vectors include plasmids, phages, cosmids, phagemids, yeast artificial chromosomes (YAC), bacterial artificial chromosomes (BAC), human artificial chromosomes (HAC), viral vectors, such as adenoviral vectors, retroviral vectors, and other DNA sequences which are able to replicate or to be replicated in vitro or in a host cell, or to convey a desired DNA segment to a desired location within a host cell.

Examples of organisms that can be hosts for vectors carrying the nucleic acid of the present invention include bacteria, yeast, flies, nematodes, animals and mammals. Examples of cells that can be hosts to vectors carrying the nucleic acids of the present invention include cells available from the American Type Culture Collection (ATCC) (Manassas, Va.).

### Transgenic Animals

In some embodiments of the invention the nucleic acids of the disclosed expression system are integrated into the genome of transgenic animals. Transgenic animals can be generated by introducing the nucleic acids disclosed herein into the germline of an animal. Methods for introducing nucleic acids into the germline of animals and generating transgenic animals, e.g. chimeric transgenics or founder lines of transgenics, are known in the art. See, e.g., Torres, R. M. and Kuhn, R., Laboratory Protocols for Conditional Gene Targeting, Qxford University Press, Oxford, U.K. (1997) and Nagy, et al., Manipulating the Mouse Embryo: A Laboratory Manual (Third Edition) Cold Springs Harbor Laboratory Press, Woodbury, N.Y. (2003). The Example provided below describes the introduction of a nucleic acid containing an inducible SHORT-RNA expression system into mouse embryonic stem cells.

Additional techniques that can be used to produce the founder lines of transgenic animals include, but are not limited to, pronuclear microinjection (U.S. Pat. No. 4,873,191), retrovirus mediated gene transfer into germ lines (Van der Putten et al., Proc. Natl. Acad. Sci., USA 82:6148, 1985), gene targeting into embryonic stem cells (Thompson et al., Cell 56:313, 1989); and electroporation of embryos (Lo, Mol. Cell. Biol. 3:1803, 1983). For a review of techniques that can be used to generate and assess transgenic animals, skilled artisans can consult Gordon (Intl. Rev. Cytol. 115:171-229, 1989), and may obtain additional guidance from, for example: Hogan et al. “Manipulating the Mouse Embryo” (Cold Spring Harbor Press, Cold Spring Harbor, N.Y., 1986; Krimpenfort et al., Bio/Technology 9:86, 1991; Palmiter et al., Cell 41:343, 1985; Kraemer et al., “Genetic Manipulation of the Early Mammalian Embryo,” Cold Spring Harbor Press, Cold Spring Harbor, N.Y., 1985; Hammer et al., Nature 315:680, 1985; Purcel et al., Science, 244:1281, 1986; Wagner et al., U.S. Pat. No. 5,175,385; and Krimpenfort et al., U.S. Pat. No. 5,175,384.

### Methods of Inducing the Inducible Expression System

When the nucleic acids described herein are introduced into eukaryotic host cells, the host cell's RNA polymerase III (Pol III) is recruited to the Pol III promoter sequence of the nucleic acid. The promoter cannot, however, initiate transcription of the short RNA encoding sequence, because of the STOP cassette that is located between the Pol III promoter and the short RNA encoding sequence. When Pol III polymerase begins moving downstream of the promoter, the polymerase encounters the termination sequence in the STOP cassette and aborts transcription before short RNA transcript synthesis begins.

Induction of short RNA expression in the system described herein is achieved by exposing the expression system to a Cre recombinase. The ability of Cre recombinase to excise loxP-flanked sequences of DNA has been extensively described. See, e.g., Guo, et al, Nature 389, 40-46 (1997) and Lakso, et al, Proc Nat'l Acad. Sci. USA 89, 6232-6236 (1992). Briefly, Cre recombinase recognizes loxP sites flanking a DNA sequence and either excises or inverts the DNA sequence between the two loxP sites. Although, loxP sequences contain two inverted 13 bp repeats, the 8 spacer nucleotides are not palindromic and provide loxP sites with an orientation. Excision occurs between two loxP sites oriented in the same direction, while inversion occurs between loxP sites that are oriented in opposite directions. A Cre-mediated excision reaction removes all the DNA between the two original loxP sites and leaves behind one loxP sequence.

In the present system, a Pol III termination sequence is flanked by two loxP sequences. Thus, in the present system, a Cre-mediated excision results in the removal of the DNA that encodes the Pol III termination signal. After removal of the termination signal, Pol III is free to bind the Pol III promoter sequence of the expression system and transcribe the short RNA encoding sequence that is downstream of the promoter. Having removed the termination signal of the STOP cassette, there remains only one loxP sequence between the promoter sequence and the short RNA encoding sequence, thereby allowing for transcription of the short RNA encoding sequence.

### Optimizing Short RNA Expression

In applications such as the synthesis of siRNA-like and micro-RNA-like gene silencing, the exact transcript sequence generated by the short RNA encoding sequence can be very important. For example, single base pair mutations can abolish the ability of a transcript to induce RNAi. It is also undesirable to include extraneous sequence in a short RNA transcript, as the extraneous sequence can also abolish gene silencing. Therefore a short RNA expression system should include features that eliminate unwanted mutations or extraneous sequence in the short RNA transcript.

The fact that Cre-mediated recombination leaves behind one 34 base pair loxP sequence between the promoter sequence and the short RNA encoding sequence can create a problem. Since Pol III promoters start transcription from between 32 and 25 base pairs downstream of the TATA box, it will frequently not be desirable to locate the TATA box of the promoter sequence upstream of the loxP site that is proximal to the promoter sequence. If the TATA box is placed upstream of the promoter-proximal loxP site, then Pol III transcription site, i.e. the (+1) site will be located inside the loxP sequence that remains after a Cre-mediated excision.

This problem can be minimized by taking advantage of the fact that the 5′ end of a loxP site has the following sequence: 5′-Adenine, Thymidine, Adenine-3′ (ATA). By introducing a thymidine reside immediately upstream of the loxP site that is proximal to the promoter sequence, a functional TATA box is produced that will remain after a Cre-mediated recombination event in the expression system.

Nonetheless, transcription can still start within the loxP even though the TATA box includes the first three nucleotides of the loxP site. For example, the transcription start site of the U6 promoter is 26 base pairs downstream of the TATA box. In an inducible expression system modified so that the TATA box includes the first three nucleotides of the remaining loxP sequence, a U6 promoter sequence will cause transcription to begin within the loxP sequence, i.e., such a transcript will include sequence encoded by the downstream terminal 5 bases of the loxP sequence.

To drive the expression of short RNA transcripts that do not begin with the terminal 5 bases of the loxP sequence, the present invention recognizes that the loxP sequence that is proximal to the short RNA encoding sequence can be mutated, so that after a recombination event, the system expresses short RNA transcripts that do not include wild-type loxP sequence. Thus, as shown in the Example below, the terminal 5 base pairs of the loxP sequence that is distal from the promoter can be mutated to encode the first 5 bases of the desired short RNA transcript. The mutation effectively creates an overlap of the mutant loxP sequence and the short RNA encoding sequence. The mutation described in the Example did not affect recombination efficiency and produced a transcript capable of inducing gene silencing.

This strategy can be generalized and adapted to different promoters and different pre-selected short RNA transcript. Once the distance from the TATA box to a transcription start site has been determined for a given Pol III promoter, the transcription start site within a remaining loxP in an expression system using that promoter can be predicted. The downstream terminal residues of the downstream loxP site in the system can then be mutated so that the mutant loxP sequence encodes the first one or more bases of a pre-selected short RNA encoding sequence, that is the downstream mutant loxP sequence and the upstream short RNA encoding sequence overlap. In this manner the system can be adapted to produce a variety of exact short RNA transcripts that do not necessarily include wild-type loxP sequence.

### Methods of Using the Inducible Expression System

The inducible expression system disclosed herein can be used in conditional, loss-of-function genetic studies in animals and cells. For example, transgenic animals whose genomes incorporate the expression system described herein can be crossed with transgenic animals carrying the Cre recombinase gene under the control of a temporally or spatially regulated promoter. Temporally regulated promoters are developmentally regulated promoters that turn on gene expression at specific stages of embryonic or animal development. Spatially regulated promoters are promoters that turn on gene expression only in defined cellular or anatomical locations, e.g., tissue-specific promoters. Many such strains of Cre transgenic mice have been developed that carry a Cre transgene under the control of a developmentally-regulated or tissue-specific promoter. One notable source of such strains is The Jackson Laboratory, Barr Harbor Me.

Even a single transgenic mouse line whose genome harbors the inducible expression systems of the present invention can be crossed with a variety of regulated Cre-expressing transgenic mice to create a variety of double transgenic mice, which are suitable for use in many conditional, loss-of-function studies. These double transgenic lines can be used to study the effects of knocking down expression of a target gene in individual tissues, e.g., to study the effects of knocking down expression of a target gene only in neural tissue or only in specific cell types. The effect of knocking down the expression of essential target genes in adult animals can be studied using double transgenics that contain a developmentally-regulated Cre gene that is only expressed in the adult animal. Similarly the role of a gene during different stages of development can be studied by using different double transgenic mice that carry the same short RNA expression construct, but different Cre transgenic constructs that express the Cre gene at different stages of development.

The expression systems described herein can also be used to study the effects of knocking down multiple gene products expressed by multiple genes, which share some genetic sequence identity. For example, an expression system coding for only one siRNA-like molecule can be used to down regulate expression of more than one gene product, provided those genes share an identical siRNA target sequence. Thus, a single nucleic acid expression system, or an organism or a cell carrying one such nucleic acid, of the present invention can be used to study the role of gene products from multiple gene family members, provided each member of the gene family shares some sequence identity with the other gene family members at the target site of for the short RNA that is inducibly expressed by the nucleic acid. The Example provided below discloses an expression system designed to produce a single short RNA transcript that down regulates several members of the A1 group of genes in the bcl-2 family of genes.

The expression system of the present invention can also be used in conjunction with other methods of conditionally delivering Cre recombinase to animals or cells harboring the nucleic acids disclosed herein. For example, cells transformed or transfected with the expression system can be exposed to exogenous Cre recombinase. The Cre protein can be delivered into the cells using any reagent suitable for the delivery of protein into a cell, e.g., liposomes or electroporation. Delivery of the Cre protein into the cell can thereby induce the recombination event that allows expression of the short RNA encoding sequence.

The inducible short RNA expression system disclosed herein is a powerful tool for conducting conditional loss of gene function experiments. Animals or cells harboring the nucleic acids disclosed herein can be induced to express the short RNA coded for by the nucleic acids, and changes in these animals or cells can be monitored. The types of changes that can be monitored include, but are not limited to, physiological changes, molecular biological changes, biochemical changes, changes in genetic expression, histological changes, gross anatomical changes, behavioral changes, changes in viability, changes in morbidity, and changes in mortality. Other changes that can be monitored include changes in compound-mediated effects on a cell or on an organism, e.g., changes in drug efficacy and/or changes in any other drug-induced effect or side effect.

### EXAMPLE

The example provides DNA construct for the inducible production of shRNAs that target the A1a, A1b, and A1d genes of the bcl-2 family. The construct was inserted into the genome of mouse embryonic stem cells, shRNA transcription was induced, and the construct was shown to selectively knockdown the expression of an A1-fusion reporter gene. (shRNAs are short hairpin RNAs that can be degraded to siRNAs that activate RNAi)

The Construct: U6lox-shA1

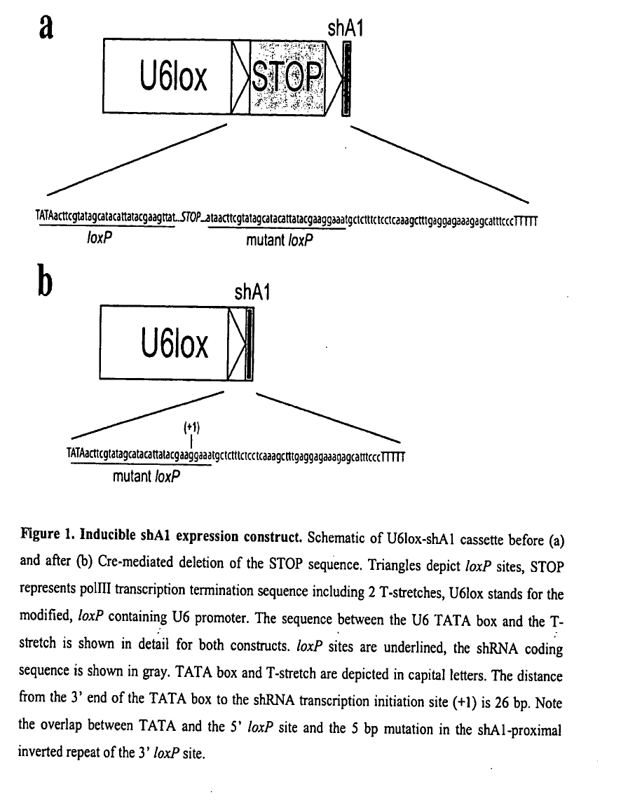

The construct used in this example included a U6 promoter sequence, a loxP-flanked STOP cassette, and an shRNA sequence. The STOP cassette included the U6 transcription termination sequence. The U6 termination sequence consisted of the wild-type run of consecutive thymidines, i.e. the T-stretch, and 190 bp of genomic DNA downstream of the T-stretch. An additional T-stretch was inserted next to the endogenous U6 T-stretch to enhance the efficiency of transcriptional termination of the STOP cassette. Insertion of the loxP-flanked STOP cassette between the U6 promoter and the shRNA gene required several adjustments in order to ensure proper shRNA transcription upon Cre-mediated deletion of the STOP sequence. Transcriptional initiation at (+1) is crucial for the precise generation of short RNAs by RNA Pol III. Deletion of the STOP cassette leaves only one loxP site at the site of its integration. If the STOP cassette were inserted after the (+1), this would result in a loxP-shRNA fusion transcript, which could interfere with proper shRNA processing and siRNA generation. To avoid transcription of the loxP site, it had to be integrated into the U6 promoter between the TATA box and (+1). Mutational analysis of the Pol III promoter suggested that this sequence could be altered without affecting the efficiency of Pol III-mediated transcription. Myslinski et al., Nucleic Acids Res. 29:2502-2509. (2001). However, since the (+1) site is located 26 bp downstream of the U6 TATA box and one loxP site comprises 34 bp, accommodation of a loxP site in the U6 promoter required the following adjustments: the first 3 bp of the loxP site (ATA) were integrated into the TATA box and the last 5 bp of the shRNA-proximal loxP site was exchanged for the first 5 bp of the shRNA coding sequence. A 5 bp mutation at the distal end on the inverted repeat was not expected to dramatically decrease recombination efficiency. FIG. 1 shows a schematic view of the inducible construct.

The entire construct is referred to as the U6lox-shA1 cassette or U6lox-shA1 construct. The shRNA sequence is referred to as, shA1, since it is directed against the bcl-2 family members A1a, A1b and A1d. Upon expression of shA1, the RNAi-processed RNA transcript produced is referred to herein as siA1.

The U6lox-shA1 cassette was cloned in three steps. First, the modified U6 promoter was PCR amplified from the U6 promoter containing plasmid pU6 (Sui, et al., Proc. Nat'l Acad. Sci. USA 99:5515-5520 (2002) using primers XbaI-U6 and U6lox-T-RI (see Table I). The 5′ primer introduced an XbaI site 5′ of the U6 promoter, the 3′ primer replaced the sequence 3′ of the TATA box with a loxP site, two T-stretches, and an EcoRI site. Second, the Pol III termination sequence was PCR amplified from C57BL/6 genomic DNA using primers U6termRI and U6term1B (see Table I), which introduced a 5′EcoRI site and a 3′ BamHI site. Third, a fragment consisting of a mutant loxP site fused to shA1 was generated by oligonucleotide synthesis of two complimentary oligomers, lox-shA1-s and lox-shA1-as (see FIG. 1 for sequence information). The annealed oligomer contained a 5′ BamHI site and a 3′ HindIII site. The three subfragments from each of the steps listed above were cloned into a modified pBS-polylinker resulting in an AscI-flanked U6lox-shA1 construct. The sequence of the U6lox-shA1 is shown in Table 2.

FIG. 1 (a) shows the U6lox-shA1 construct before a Cre-mediated excision of the termination sequence, and FIG. 1 (b) shows the U6lox-shA1 construct after a Cre-mediated deletion of the termination sequence. Triangles are loxP sites, the STOP rectangle is U6 termination sequence comprising two T-stretches and 190 bp of wild-type genomic sequence immediately downstream of the genomic U6 T-stretch, U6lox is a modified U6 promoter sequence containing a loxP site. Sequence from TATA box to the T-stretch following shA1 sequence is shown below each construct (the omitted wild-type U6 termination sequence is marked with “STOP”). The distance from the 3′ end of the TATA box is to the shRNA transcription initiation site (+1) is 26 bp. FIG. 6 shows the overlap between TATA and the 5′ end of the loxP sequence, and it also shows the overlap between the upstream 5 bp of the shA1 encoding sequence and shA1 proximal terminus of the mutant loxP.

Insertion of the Construct into HPRT Deficient HM1 Embryonic Stem Cells

As a first step in generating transgenic mouse strain that allows ubiquitous induction of shA1-mediated RNAi upon Cre-mediated recombination in a defined genetic locus, the U6lox-shA1 construct was targeted into the X-linked hypoxanthine phosphoribosyltransferase (HPRT) locus by homologous recombination in ES cells. This approach takes advantage of the fact that HPRT-deficient HM-1 ES cells permit extremely efficient selection of transgenes inserted into the HPRT locus. Thompson, et al., Cell 56:313-321 (1989). HM-1 ES cells lack the HPRT promoter and exons 1 and 2. Only reconstitution of the disrupted HPRT locus by gene targeting confers resistance to HAT selection. Hence, virtually every HAT-resistant ES cell colony carries the targeted HPRT allele. A targeting vector that allows the insertion of transgenes into HM-1 ES cells has been described previously (pMP-8SKB, (Bronson et al., 1996)). A modified version of this vector referred to as pMP-10, has been developed, which can be linearized with SwaI, SbfI or SgfI and harbors two additional unique restriction sites (AscI and PmeI) to insert the transgene of choice. The U6lox-shA1 cassette was inserted into the AscI restriction site of pMP-10 in the same transcriptional orientation as the HPRT gene. The targeting vector was linearized with SwaI and transfected into HM-1 ES cells.

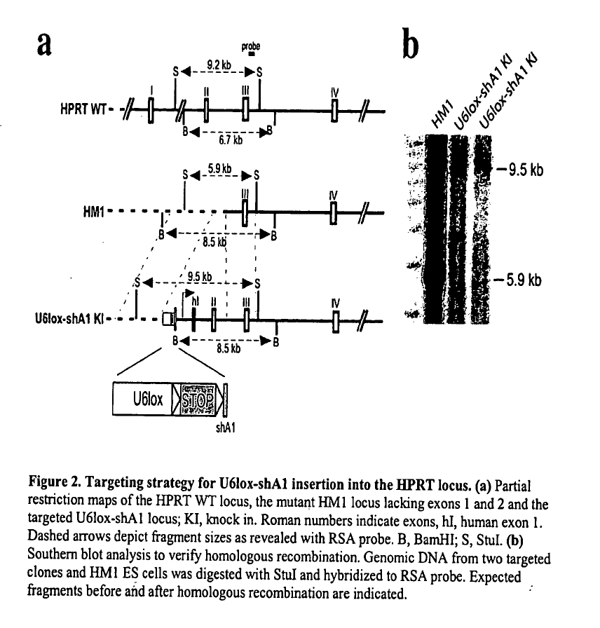

The targeting strategy is shown in FIG. 2a. FIG. 2a shows a partial restriction map of the HPRT wild-type genomic locus (HPRT WT), below which is a partial restriction map of the HM1 mutant HPRT genomic locus (HM1), and below both of which, is a partial restriction map showing the insertion, i.e. Knock-In, of the U6lox-shA1 construct into the HM1 mutant HPRT locus (U6lox-shA1 KI). HPRT exons are shown as boxes with roman numerals above them. StuI restriction sites are marked by a capital S.

FIG. 2b is a Southern Blot confirming insertion of the U6lox-shA1 construct. The integrity of HAT-resistant colonies was confirmed by Southern blotting using a StuI digest and probe RSA. RSA is shown in FIG. 2a above the general location of its binding site near HPRT exon III. Two independent ES cell clones were injected into C57BL/6 blastocysts.

### Testing the Construct

The ability of the construct to effect Cre-mediated induction of shRNA expression and subsequently knock down A1 expression was tested in transgenic ES cells. Endogenous A1 expression is barely detectable in ES cells. Therefore, to increase measurable A1 signal, a transgene encoding an A1-IRES-EEGFP reporter protein was introduced into targeted ES cells. A1 cDNA was fused to DNA containing an internal ribosomal entry site (IRES) followed by EEGFP cDNA. Expression of this fusion construct results in a bicistronic mRNA encoding A1 and EEGFP. Degradation of this construct by siA1-mediated mRNA degradation was predicted to result in loss of both A1 and EEGFP expression.

The coding sequence of the mouse A1d gene was PCR-amplified from splenic cDNA using primers A1d-X and A1d-B (see Table I), which introduced a 5′ XhoI site and a 3′ BamHI site. The PCR fragment was then subcloned into BamHI/XhoI-digested pIRES2-EEGFP (Clontech, Palo Alto, Calif.) to generate the A1-IRES-EGFP fusion construct.

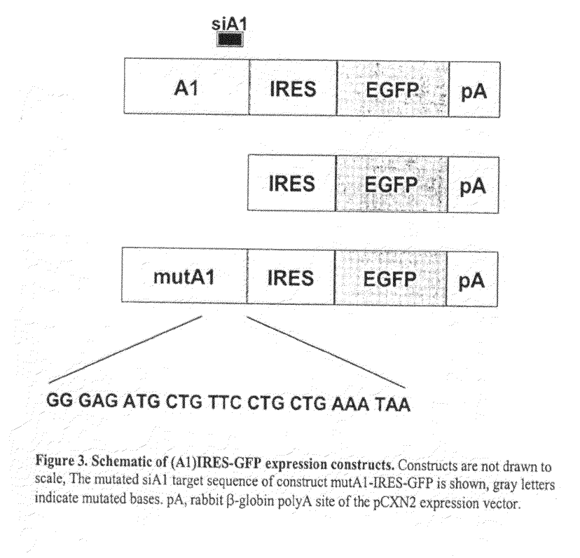

To test the sequence specificity of siA1, a second, mutated A1 expression construct (mutA1-IRES-EGFP) was cloned into pIRES2-EEGFP. The mutA1 cDNA contains 6 conservative mutations at the siA1 target site (see FIG. 3) and was generated by PCR amplification using primers A1d-X and mutA1d-B (see Table I). A1-IRES-EGFP constructs were subcloned into the neoR selectable marker containing expression vector pCXN2. Niwa, et al., Gene 108:193-199 (1991).

A1-IRES-EGFP, mutA1-IRES-EGFP and IRES-EGFP fragments were excised from the respective pIRES2-EEGFP vectors using XhoI and NotI and inserted into an XhoI site 3′ of the chicken b-actin promoter of pCXN2. Expression vectors were SalI-linearized and transfected into U6lox-shA1 ES cells. Stable integrants were selected with G418 starting 2 days after transfection. Single G418-resistant ES cell colonies were analyzed for EGFP expression in order to confirm expression of the reporter transgene.

FIG. 3 depicts the three fusion constructs used to verify specific RNAi-mediated gene silencing by shA1. A1 box represents the A1 cDNA sequence, the IRES box represents the internal ribosome entry site sequence, EEGFP box represents the EEGFP gene, and pA box represents the polyadenylation (poly A) site from the pCNX2 expression vector. The mutA1 box represents the mutated A1 cDNA; gray letters in the sequence below the mutA1 box indicate mutated bases. The siA1 box represents predicted product of RNAi processed shA1 transcript, the siA1 box is depicted above the siA1 target site.

### Cre-Mediated Induction of RNAi in ES Cells

EGFP+ clones of each transgenic ES cell line were transduced with a Cre expressing adenovirus in order to delete the loxP-flanked STOP cassette and induce shRNA expression. See, e.g., Bassing, et al, Cell 109 Suppl:S45-55 (2002). Untransduced cells served as negative control. Seven days after transduction, ES cells were analyzed for EGFP expression by FACS analysis.

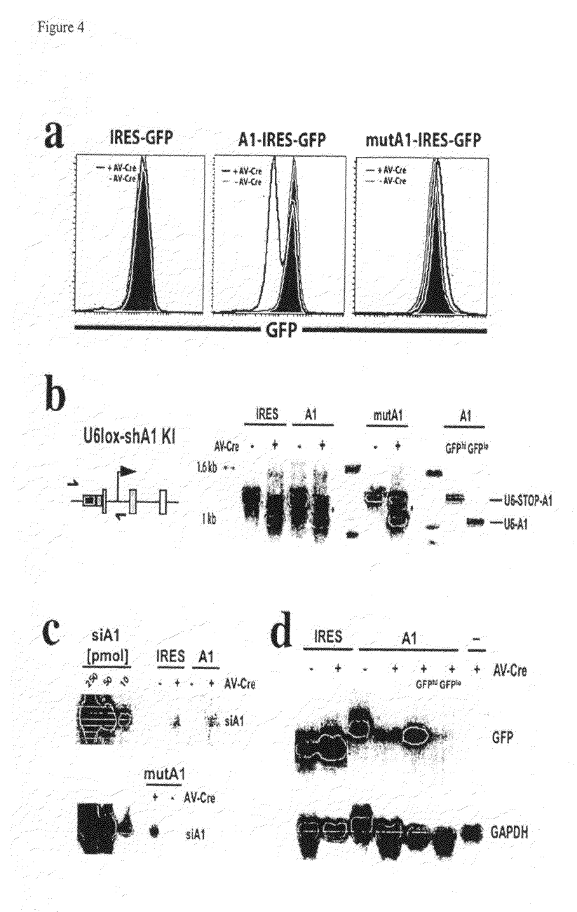

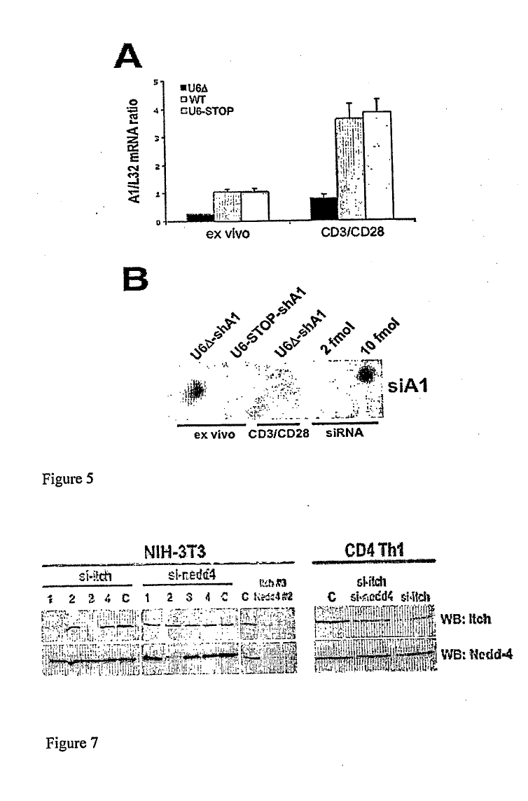

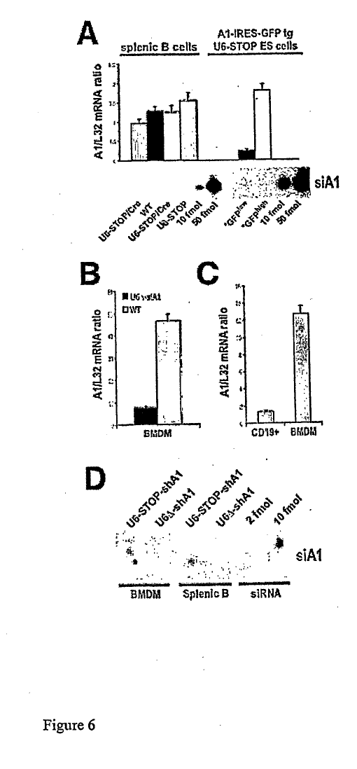

FIG. 4A shows that only ES cell clones that were exposed to Cre and carried the perfectly complementary A1-IRES-EGFP transgene showed downregulation of EGFP expression, demonstrating sequence-specific and inducible RNAi in U6lox-shA1 ES cells. FIG. 4A depicts the results of FACS analysis of EGFP expression in transduced (open histograms) or untransduced ES cells (shaded histograms). The respective EGFP transgene is indicated, and AV-Cre stands for Cre expressing adenovirus.

The fact that EGFP downregulation occurred only in ˜60% of cells likely reflects incomplete deletion of the STOP cassette. This was confirmed by PCR analysis of genomic DNA isolated from total cell lysate or subpopulations that were sorted according to EGFP expression levels. Deletion of the STOP cassette occurred exclusively in cells showing EGFP downregulation, i.e. the EGFP-low cells, as shown in FIG. 4B.

FIG. 4B shows a schematic of the targeted HPRT locus. Half-arrows depict primers hHPRT-pro and HPRT-SAH (see Table I) flanking the inserted U6lox-shA1 cassette. The arrow represents the human HPRT promoter, the gray box depicts human exon 1, the white box mouse exon 2; map is not drawn to scale. PCR results are shown for transduced and untransduced ES cells transgenic for IRES-EGFP (IRES), A1-IRES-EGFP (A1) or mutA1-IRES-EGFP (mutA1). A1-IRES-EGFP transgenic ES cells were sorted according to EGFP expression levels. DNA from EGFPhigh cells and EGFPlow cells was subjected to PCR. The expected sizes for PCR fragments before (U6-STOP-A1) and after deletion of the Pol III STOP cassette (U6-A1) are indicated. The asterisk indicates a fragment resulting from a DNA hybrid of one U6-STOP-A1strand and one U6-A1 strand.

Importantly, FIGS. 9C and 9D show that similar levels of Cre-mediated deletion and concomitant siRNA generation were detected in all Cre-treated ES cell lines, emphasizing the specificity of siA1 for A1-IRES-EGFP mRNA. To determine the extent of mRNA degradation, EGFP containing mRNA levels were analyzed by Northern blotting using a probe specific for EEGFP. The results of Northern blot analysis of transduced and untransduced ES cells carrying the indicated transgene are shown in FIGS. 9C and 9D. 20 mg of total RNA were loaded per lane. Synthetic double-stranded siRNA of identical sequence were loaded in the amounts indicated above the siA1 lanes of FIG. 4C to estimate siRNA expression levels. The size of the detected mRNA differed depending on the presence or absence of the A1 cDNA. Detection of GAPDH mRNA served to normalize for loading differences. EGFP mRNA levels were strongly reduced in total cell lysate and the remaining mRNA is likely to originate from cells that have not undergone deletion of the STOP cassette. Indeed, when cells were sorted according to EGFP expression, A1-IRES-EGFP mRNA was barely detectable in EGFPlow cells and image quantification showed a >10 fold reduction of mRNA when compared to EGFPhigh cells. No mRNA reduction could be observed in untransduced A1-IRES-EGFP transgenic ES cells or in IRES-EGFP control samples. These data demonstrate that a single copy of the U6lox-shA1 cassette mediates efficient, sequence-specific and tightly regulated suppression of A1 in vitro.

TABLE 1
Primers for polymerase chain reaction (PCR)

| | | | |
|---|---|---|---|
| Name | Sequence (5′-3′) | Location | → |

| | | | |
|---|---|---|---|
| LAH53 | GGACCTCCATCTGCTCTTATTT | 5′ of DQ52 | s* |
| CDR3-PE | GGTCTATTACTGTGCAAGTTGG | CDR3 of VPE | as |
| U6termRI | TGTGAATTCGTTCCTCAGAGGAACTGA | 3′ of U6 gene | s |
| U6term1-B | TGTGGATCCCCCGGGCGTGGCTTGGTGGTACACCTC | 3′ of U6 gene | as |
| XbaI-U6 | GACTCTAGATCCGACGCCGCCATCTCTAG | U6 promoter | s |
| U6loxT-RI | TGCGAATTCAAAAATCGCAAAAACGTAATAACTTCGTATA | U6 promoter | as |
| | AGTATGCTATACGAAGTTATAGTCTCAAAACACACAATTA | | |
| | CTTAC | | |
| A1d-X | TGCTCGAGATGTCTGAGTACGAGTTCATGCATATC | A1d cDNA | s |
| mutA1-B | CTGGATCCTTATTTCAGCAGGAACAGCATCTCCCATATCT | A1d cDNA | as |
| | G | | |
| A1d-B | CTGGATCCTTACTTGAGGAGAAAGAGCATTTC | A1d cDNA | as |
| HPRT-SAH | TTCCTAATAACCCAGCCTTTG | pMP-10 SAH | s |
| hHPRT-pro | GTGATGGCAGGAGATTTGTAA | hHPRT promoter | as |

Abbreviations in Table 1:
s, sense strand;
as, antisense strand

TABLE 2
Sequence of the U6lox-shA1 construct

5′-tccgacgccgccatctctaggcccgcgccggccccctcgcacagact
tgtgggagaagctcggctactcccctgccccggttaatttgcatataata
tttcctagtaactatagaggcttaatgtgcgataaaagacagataatctg
ttctttttaatactagctacattttacatgataggcttggatttctataa
gagatacaaatactaaattattattttaaaaaacagcacaaaaggaaact
caccctaactgtaaagtaattgtgtgttttgagactataacttcgtatag
catacattatacgaagttattacgtttttgcgatttttgaattcgttcct
cagaggaactgacaagcaccctaacatcctattggaggctcactcacgtt
ttttctattttgtttcttgacagcagagctcgttgctcactgtatagctc
aggttggcctgacactgatgaggttctccagtgactgcctctacctacct
actgggatgacagaggtgtaccaccaagccacgcccgggggatccataac
ttcgtatagcatacattatacgaaggaaatgctctttctcctcaaagctt
tgaggagaaagagcatttcccttttt-3′

The nucleotide sequence in Table 2 encodes the following functional units (numbering begins at the 5′ end):

1-282: U6 promoter upstream of TATA box

283-287: TATA box

284-317: loxP

318-530: termination sequence starting with additional TTTT

543-577: mutant loxP

572-end: shA1 hairpin plus T-stretch

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid molecule comprising:
an RNA polymerase III promoter sequence;
a short RNA encoding sequence comprising a transcription initiation site;
a loxP-flanked STOP cassette comprising an RNA polymerase III-specific termination sequence, a first loxP sequence, and a second loxP sequence, wherein (i) each of the two loxP sequences comprises a spacer region, (ii) the termination sequence is disposed between the first and second loxP sequences, and (iii) the termination sequence is disposed between the promoter sequence and the transcription initiation site of the short RNA encoding sequence in the nucleic acid molecule.

2. The molecule of claim 1, wherein each of the loxP sequences comprises one or more mutations in its spacer region.

3. The molecule of claim 1, wherein the first loxP sequence is a wild-type loxP sequence.

4. The molecule of claim 1, wherein the second loxP sequence is a mutant loxP sequence.

5. The molecule of claim 1, wherein
the second loxP sequence is closer to the short RNA encoding sequence that the first loxP sequence;
the second loxP sequence comprises a distal terminal sequence and a proximal terminal sequences, wherein the spacer region is disposed between the distal and the proximal terminal sequence, the distal terminal sequence is closer to the termination sequence than the spacer region, and the proximal terminal sequence is closer to the shRNA encoding sequence than spacer region;
the second loxP proximal terminal sequence overlaps with 1 to 10 nucleotides of 5′ end of the short RNA encoding sequence; and
the 1 to 10 nucleotides of the 3′end of the second loxP proximal terminal sequence consists of the 5′end of the short RNA encoding sequence.

6. The molecule of claim 1, further comprising a thymidine nucleotide immediately preceding the upstream terminal sequence of the first loxP, wherein the first loxP is upstream of the termination sequence.

7. The molecule of claim 1, wherein the RNA polymerase III promoter sequence comprises genomic sequence of the small nuclear RNA U6 promoter or a functional equivalent thereof.

8. The molecule of claim 7, wherein:
the termination sequence comprises genomic sequence downstream of the small nuclear RNA U6 transcription termination signal.

9. The molecule of claim 8, wherein the termination sequence is a modified U6 transcription termination sequence comprising:
between 1 to 20, inclusive, additional thymidine nucleotides disposed immediately adjacent to the wild-type U6 thymidine termination signal; and
between 1 to 190, inclusive, additional nucleotides of animal genomic sequence that is immediately downstream of the thymidine termination sequence of wild-type small nuclear RNA U6 gene.

10. The molecule of claim 8, wherein the termination sequence further comprises one or more additional RNA Polymerase III termination signals.

11. The molecule of claim 1, wherein the short RNA encoding sequence encodes a transcript with fewer than 30 nucleotides.

12. The molecule of claim 1, wherein the molecule comprises a sequence selected from the group consisting of: SEQ ID NOs: 1 to 7.

13. A transgenic animal whose genome comprises the nucleic acid molecule of claim 1.

14. The transgenic animal of claim 13, further comprising a nucleic acid molecule encoding a Cre recombinase.

15. The transgenic animal of claim 14, wherein expression of the Cre recombinase is developmentally regulated.

16. The transgenic animal of claim 13, wherein expression of the Cre recombinase is tissue-specific.

17. The animal of claim 13, wherein the animal is selected from the group consisting of a mouse, a rat, a guinea pig, a goat, a pig, a monkey, a baboon, a chimpanzee, a cow, a rabbit, a sheep, a dog, a cat, a hamster, a chicken, and a frog.

18. A eukaryotic cell comprising the nucleic acid molecule of claim 1.

19. The cell of claim 18, wherein the cell is an animal cell.

20. The cell of claim 18, wherein the cell is a mammalian cell.

21. The cell of claim 19, wherein the cell is an embryonic stem cell.

22. The cell of claim 18, further comprising a nucleic acid molecule encoding a Cre recombinase gene.

23. The cell of claim 18, further comprising a Cre recombinase protein.

24. A method of making an inducible short RNA expression system, the method comprising linking two or more nucleic acids to produce the nucleic acid of claim 1.

25. A method of making a transgenic animal comprising:
introducing the molecule of claim 1 into the genome of an embryonic stem cell;
introducing the embryonic stem cell into an embryo;
implanting the embryo in an animal capable of carrying the embryo to term; and
allowing the embryo to come to term, thereby generating a transgenic animal.

26. The method of claim 25, wherein:
the molecule of claim 1 is introduced into the genome of an oocyte;
the oocyte is fertilized to produce an embryo;
the embryo is implanted in an animal capable of carrying the embryo to viability; and
the embryo is allowed to become a viable animal, thereby generating a founder transgenic animal.

27. The method of claim 25, wherein the method generates a chimeric transgenic animal, and further comprising:
crossing the chimeric transgenic animal to another animal of the same species to generate a founder transgenic animal whose genome includes the molecule of claim 1.

28. A method of making an animal cell containing an inducible short RNA expression, the method comprising:
transfecting a cell with the molecule of claim 1.

29. The method of claim 28, wherein the cell is a cell from any one of the following animals: a human, a mouse, a rat, a guinea pig, a goat, a pig, a monkey, a baboon, a chimpanzee, a cow; a horse, a rabbit; a sheep, a chicken, a dog, a cat, a frog, or a fish.

30. A method of evaluating gene function in a cell, the method comprising:
providing the cell of claim 18;
inducing transcription of the short RNA encoding sequence; and
monitoring changes in the cell.

31. A method of evaluating gene function in an organism, the method comprising:
providing the transgenic animal of claim 13;
inducing transcription of the short RNA encoding sequence; and
monitoring changes in the organism.

32. A method of treating a patient, the method comprising:
administering the molecule of claim 1 into a patient in need of having expression of one or more genes reduced, wherein the short RNA encoding sequence encodes a transcript designed to reduce expression of the one or more genes the patient is in need of reducing.

33. The method of claim 32, wherein the method comprises administering the molecule in the cell of claim 18.

34. A method of identifying a candidate RNAi effector with reduced activity in T-cells, the method comprising:
administering or inducing expression of siRNA in a T-cell and a control cell;
evaluating expression of an mRNAs or protein in the T-cell and the control cell; and
identifying an mRNA or protein (a) with a reduced expression level or (b) that is differently modified in the T-cell relative to control,
wherein the control cell is not a mature lymphocyte and an mRNA or protein with reduced levels or that is differently modified in the T-cell relative to control is a candidate RNAi effector with reduced activity in T-cells.

35. A method of identifying a candidate inhibitor of RNAi in T-cells, the method comprising:
administering or inducing expression of siRNA in a T-cell and a control cell;
evaluating expression of an mRNA or protein in the T-cell and the control cell; and
identifying an mRNA or protein (a) with an increased expression level or (b) that is differently modified in the T-cell relative to control;
wherein the control cell is not a mature lymphocyte and an mRNA or protein with reduced levels or that is differently modified in the T-cell relative to control is a candidate inhibitor of RNAi in T-cells.

36. A method of identifying a missing RNAi effector or inhibitor of RNAi in T-cells, the method comprising:
identifying a candidate missing RNAi effector or candidate inhibitor of RNAi by performing the method of claim 34; and
(i) in one or more T-cells, (a) introducing the identified candidate RNAi effector or (b) modifying the identified candidate RNAi effector, and subsequently determining if (a) or (b) increases RNAi efficiency in the one or more T-cells, wherein an increases RNAi efficiency is an RNAi effector with reduced activity in T-cells;
(ii) introducing or modifying the identified candidate inhibitor of RNAi in a cell, and subsequently determining if it reduces RNAi efficiency in the cell, wherein a candidate that reduces RNAi efficiency in the cell is an inhibitor of RNAi in T-cells; or
(iii) inactivating the identified candidate inhibitor in a T-cell, and subsequently determining if inactivation increases RNAi efficiency in the T-cell, wherein an inactivated candidate inhibitor that increases RNAi efficiency in the T-cell is an inhibitor of RNAi in T-cells.
